(19) United States
(12) Patent Application Publication
(10) Pub. No.: US 2019/0046813 A1
(43) Pub. Date: Feb. 14, 2019

(54) Systems and Methods for Radiation Treatment Planning

(71) Applicant: Suzhou Evidance Medical Technologies Inc., Suzhou, Jiangsu (CN)

(72) Inventors: Yin Zhou, Suzhou, Jiangsu (CN); Cheng Li, Suzhou, Jiangsu (CN); Meng Zhu, Suzhou, Jiangsu (CN); Haihang Jiang, Suzhou, Jiangsu (CN); Yu Sheng, Suzhou, Jiangsu (CN); Zizhuo Wang, Suzhou, Jiangsu (CN); Wentao Zhang, Suzhou, Jiangsu (CN)

(21) Appl. No.: 16/074,746

(22) PCT Filed: Feb. 2, 2017
(86) PCT No.: PCT/CN2017/072798

§ 371 Date: Aug. 1, 2018

Related U.S. Application Data
(63) Continuation of application No. PCT/CN2016/073155, filed on Feb. 2, 2016.

(51) Int. Cl. A61N 5/10; H04L 29/08; H04L 29/06

(52) U.S. Cl. CPC A61N 5/103; H04L 67/42; H04L 29/08

(57) ABSTRACT

The present disclosure relates to improved workflows, methods and systems for the generation of optimized radiation treatment plans. In some embodiments, cloud servers and remote devices such as a wireless device are used.

### TECHNICAL FIELD

The present disclosure relates generally to radiation therapies, and more particularly, to an improved workflow for the generation of optimized radiation treatment plans. In some embodiments, cloud servers and remote devices (e.g., wireless mobile devices) are used.

### BACKGROUND

It is estimated that each year 12 million cases of cancer are diagnosed, and that every year 7.6 million patients die of cancer. Radiation therapy is widely used to treat localized cancer. In a typical application, a radiation delivery system has an ionizing radiation device mounted to a movable (rotabable) gantry. The radiation delivery system controls the motion/rotation of the radiation device to direct the center of an ionizing radiation beam to a specific point in space commonly referred to as the “machine isocenter.” During radiation therapy, a patient is positioned so that the patient's tumor is usually located at the machine isocenter throughout treatment.

Radiation is typically delivered to a patient during a radiation therapy session in accordance with a session plan. A session plan typically specifies, for each of one or more “treatment fields,” such information as the gantry position, which determines the path that radiation energy will take to the tumor during the treatment field; collimator settings that determine the shape and cross-sectional area of the radiation energy beam; the intensity level of the radiation beam; and a duration that determines for how much time radiation energy will be delivered during the field.

A plan is typically prepared using determinants such as the tumor's mass, volume, shape, orientation, location in the body, and proximity to different organs and other anatomical structures; information about radiation energy intended to be delivered to the tumor in foregoing radiation therapy sessions, as well as other approaches previously used to treat the tumor.

Current workflow of radiation treatment planning is divided into several stages, including computerized tomography (CT) simulation, tumor target volume and critical organs contouring, prescription (of radiation dose), normal tissue dose constraints, radiation plan design, plan optimization, plan evaluation, plan re-optimization (if required), further evaluation and re-optimization (if required), and final plan approval/verification. Some of the process steps are typically performed by a physician (e.g., radiation oncologist) and/or a physicist (e.g., therapeutic medical physicist), such as the contouring and prescription steps. Some we performed by a medical dosimetrist who designs a treatment plan by means of computer and/or manual computation to determine a treatment field technique that will deliver the prescribed radiation dose. In addition, the contouring and optimized plan must be viewed and approved by a supervisory or senior physician. A specialized workstation is required to carry out each of these steps where the working physician, physicist, dosimetrist or supervisor uses one or more dedicated software provided by the vendor. Subsequently, data generated from the workflow is circulated within a Local Area Network (LAN) using File Transfer Protocol (FTP) or Digital Imaging and Communications in Medicine (DICOM) protocol.

As such, the current workflow process requires the use of a stationary work station to execute almost every task in the workflow. The user has little freedom in choosing the work location or work time. If the supervising physician is not available to review and approve treatment plans, the user must halt the workflow until such plans can be reviewed and approved, which reduces efficiency and smoothness of the operation and delays treatment of the patients.

Furthermore, users operations are limited by the difficulties associated with integration of various types of software and hardware. For example, different steps in the workflow use different software programs, which are operating system (OS) dependent and are tailored to a particular piece of hardware equipment, as the vendor of the hardware equipment intended. As such, system integration, such as transferring treatment data between different steps of the workflow may be difficult because the software programs are not compatible with each other.

Intensity modulated radiation therapy (IMRT) has been increasingly used for targeted, precision X-ray radiation therapy. In IMRT, the multi-leaf collimator is operated to control the leaves such that different parts of the target region receive different amount of doses, since treatment field may be inhomogeneous and complex shaped dose distributions may be realized. In such cases, information regarding the different desired dose for different parts of the target region, dose constraints of normal tissue, and the mechanical information regarding the constraints for the operation of the collimator (e.g., orientation of collimator, leaves' speed, etc.) are incorporated into the objective function during treatment planning. In order to obtain a precise treatment plan that matches the oncologist's prescription, dosimetrists must iteratively adjust various parameters during optimization. For example, the dosimetrist must first set the radiation beam angle, set specific objective parameters for dose distribution using single dose value, dose-volume point, dose-volume charts and other tools, and the weights of each of objective parameters and then use some commercial treatment planning system software such as Pinnacle to generate the treatment plan. If the plan does not meet the oncologist's expectation, then the dosimetrist must adjust various parameters by repeated “trial-and-error” cycles in the optimization software, until the acceptable treatment plan in compliance with the expectation is found. This exploration process is extremely time and labor consuming in clinical practice. For some tumors (such as head and neck), the process needs up to a week and a great deal of dosimetrists' workload to complete, which may affect the treatment plan quality and delay the patient treatment. Especially for developing countries such as China, well-trained dosimetrists are scarcely available, negatively impacting the healthcare system.

Volumetric modulated arc therapy (VMAT) is another X-ray radiation technique that allows the simultaneous variation of three parameters during treatment delivery, i.e., gantry rotation speed, treatment aperture shape via movement of MLC leaves and dose rate. VMAT differs from IMRT because it delivers the dose to the whole volume while the gantry is rotating, rather than from several fixed beams with different angles. Therefore, VMAT is able to provide better plan quality and much faster dose delivery but more complicated optimization and delivery process comparing with IMRT.

Intensity modulated proton therapy (IMPT) implies the electromagnetic spatial control of well-circumscribed “pencil beams” of protons of variable energy and intensity. Proton pencil beams take advantage of the charged-particle Bragg peak—the characteristic peak of dose at the end of range—combined with the modulation of pencil beam intensity variables to create target-local modulations in dose that achieves the dose objectives. IMPT improves on X-ray intensity modulated beams (IMRT) with dose modulation along the beam axis as well as lateral, in-field, dose modulation. The clinical practice of IMPT further improves the healthy tissue vs target dose differential in comparison with X-rays and thus allows increased target dose with dose reduction elsewhere. However, the wide application of IMPT is limited because IMPT requires not only the highest precision tools but also the highest level of system integration of the services required to deliver high-precision radiotherapy.

Thus, current workflow is constrained by location and availability of the work station, as well as other factors such as unavailability of the supervising physician, lack of compatibility between different types of software and hardware, and lack of well-trained dosimetrists. As such, there is a need for improved methods and systems for generating radiation treatment plans that are both effective and efficient.

### SUMMARY

The present disclosure is directed to methods and systems for radiation treatment planning.

In one aspect, a method is provided, comprising:

collecting at a central server image data of a tumor and surrounding anatomic structures;

accessing the image data from a remote device, the remote device having an interface for processing the image data into contours of tumor target volume and critical organs;

providing, via the interface of the remote device, a radiation prescription value based on the image data;

processing, at the central server, the contours and the prescription value to generate a radiation treatment plan; and

receiving the radiation treatment plan at the remote device.

In some embodiments, the remote device is a device with a pure web-browser based interface and/or a wireless mobile device.

In certain embodiments, the collecting step comprises collecting the image data from, e.g., one or more of computerized tomography (CT), positron emission tomography (PET), ultrasound, single-photon emission computed tomography (SPECT) or magnetic resonance imaging (MRI) machine. Preferably the collecting step further comprises uploading the image data to a local server that synchronizes with a mirror node on the central server. More preferably the central server is a cloud server for storing index information associated with the radiation treatment plan, wherein the cloud server is connected to the remote device, allowing access to the cloud server through the interface.

In some embodiments, the prescription value comprises one or more of radiation dose, hard constraint of the critical organs' dose-volume histogram (DVH), maximal dose limit, minimal dose limit, mean dose limit, and effective uniform dose (EUD).

In certain embodiments, the accessing step comprises generating contours of tumor target volume and critical organs in the image data via the interface. The generating step may optionally comprise auto-generating the contours using an automatic segmentation software and modifying the auto-generated contours via the interface. The interface may be configured to add to, or remove from, the image data a region of interest (ROI). The interface may also be configured to add to, or remove from, the image data a point of interest (POI). In select embodiments, the accessing step further comprises providing a contouring input device selected from, e.g., one or more of a finger, a pen and a mouse. In certain embodiments, in the accessing step, operations supported comprise, e.g., one or more of zoom in, zoom out, select, move, copy, paste, cut object, resize object, and change contrast.

In certain embodiments, the processing step comprises reconstructing 3D volume and surface representation of the target volume and critical organs.

The method, in some embodiments, further comprises generating, based on the treatment plan, an evaluation index from, e.g., one or more of: two dimensional or three dimensional isodose distribution and/or curve in a region of interest, a dose-volume histogram (DVH) for the tumor target volume and critical organs contoured, Conformality Index (CI), Heterogeneity Index (HI) of the target volume, Tumor Control Probability (TCP), and Normal Tissue Complication Probability (NTCP). The treatment plan may be forwarded to a third party remote device for approval, together with the evaluation index. The third party remote device may be notified by one or more of: highlighted message, instant messaging tool, beep, short recorded sound track, automatic phone call and voice mail. The approved treatment plan can be transmitted to a radiation treatment machine for execution to carry out a radiation modality. In certain embodiments, the radiation modality is selected from, e.g., intensity-modulated radiation therapy (IMRT), volumetric modulated are therapy (VMAT), intensity modulated proton therapy (IMPT) and brachytherapy.

In certain embodiments, the processing step comprises generating the radiation treatment plan using a software module. For example, the processing step can comprise exporting the contours to a treatment planning system (TPS) to generate the radiation treatment plan.

Also provided herein is a system having computer program code stored on a non-transitory computer readable medium for generating a radiation treatment plan, comprising:

a central server having a processor unit for storing and processing image data of a tumor; and

a remote device connected to the central server, the remote device having an interface for accessing the image data and processing the image data into contours of tumor target volume and critical organs, wherein the interface is configured to receive a treatment prescription value and transmit the prescription value to the central server;

wherein the remote device is configured to interact with the central server from a remote location, and wherein the central server has one or more algorithms for in processing the contours and the prescription value to generate a radiation treatment plan.

In various embodiments, the remote device is a device with a pure web-browser based interface and/or a wireless mobile device. In certain embodiments, the remote device supports one or more operations selected from, e.g., zoom in, zoom out, select, move, copy, paste, cut object, resize object, and change contrast.

In some embodiments, the central server is a cloud server for storing index information associated with the radiation treatment plan, wherein the cloud server is connected to the remote device, allowing access to the cloud server through the interface.

The system can further comprise an imaging equipment for generating the image data, wherein preferably the imaging equipment comprises one or more of a computerized tomography (CT), a positron emission tomography (PET), an ultrasound, a single-photon emission computed tomography (SPECT) and a magnetic resonance imaging (MRI) machine.

The system in some embodiments can additionally include a local server for storing the image data, wherein the local server is connected to the remote device and accessible through the interface. Preferably the content of the local server is synchronized with the central server.

In some embodiments, the interface is configured to add to, or remove from, the image data a region of interest (ROI). The interface can also be configured to add to, or remove from, the image data a point of interest (POI).

In some embodiments, the prescription value comprises, e.g., one or more of radiation dose, hard constraint of the critical organs' dose-volume histogram (DVH), maximal dose limit, minimal dose limit, mean dose limit, and effective uniform dose (EUD).

In certain embodiments, the central server is configured to reconstruct three dimensional volume and surface representation of the target volume and critical organs. The central server may also be configured to generate, based on the treatment plan, an evaluation index from, e.g., one or more of: two dimensional or three dimensional isodose distribution and/or curve in a region of interest, a dose-volume histogram (DVH) for the tumor target volume and critical organs contoured, Conformality Index (CI), Heterogeneity Index (HI) of the target volume, Tumor Control Probability (TCP), and Normal Tissue Complication Probability (NTCP). In some embodiments, the central server is further configured to forward the treatment plan to a third party remote device for approval, together with the evaluation index. The system can further comprise a radiation treatment machine for receiving and executing the approved treatment plan to carry out a radiation modality. In some embodiments, the radiation modality is selected from intensity-modulated radiation therapy (IMRT), volumetric modulated arc therapy (VMAT), intensity modulated proton therapy (IMPT) and brachytherapy.

The system, in some embodiments, can further include a contouring input device selected from, e.g., one or more of a finger, a pen and a mouse.

In certain embodiments, the system further comprises a third party remote device for reviewing and approving the radiation treatment plan. The third party remote device may comprise a notification function selected from, e.g., one or more of: highlighted message, instant messaging tool, beep, short recorded sound track, automatic phone call and voice mail.

### BRIEF DESCRIPTION OF THE DRAWINGS

Illustrative, non-limiting exemplary embodiments will be more clearly understood from the following detailed description taken in conjunction with the accompanying drawings.

FIGS. 1A-C illustrate exemplary cloud based platforms for generating radiation treatment plans.

FIG. 2 illustrates a high level overview of an exemplary cloud based system for generating a radiation treatment plan.

FIGS. 3A-3B illustrate examples of a cloud based system for generating radiation treatment plans.

FIG. 4 illustrates some of the functions an exemplary cloud based system can provide to assist radiation treatment.

FIG. 5 illustrates an exemplary cloud server optimizing a treatment plan.

FIG. 6 illustrates an exemplary cloud server modifying treatment plans according to tumor sizes.

FIGS. 7A and 7B illustrate exemplary region of interest (ROI) being modified by a user.

FIGS. 8A-8B illustrate exemplary auto placement of ROIs by an exemplary cloud server.

FIG. 9 illustrates exemplary treatment plan optimization based on GPU.

FIG. 10 illustrates an exemplary cloud based Monte Carlo simulation for generating radiation treatment plans.

FIG. 11 illustrates an exemplary flowchart for generating an optimized radiation treatment plan.

### DETAILED DESCRIPTION

Various exemplary embodiments will be described more fully hereinafter with reference to the accompanying drawings, in which some exemplary embodiments are shown. The present inventive concept may, however, be embodied in many different forms and should not be construed as limited to the exemplary embodiments set forth herein. Rather, these exemplary embodiments are provided so that this disclosure will be thorough and complete, and will fully convey the scope of the present inventive concept to those skilled in the art. In the drawings, the sizes and relative sizes of layers and regions may be exaggerated for clarity. Like numerals refer to like elements throughout.

It will be understood that, although the terms first, second, third, etc. may be used herein to describe various elements, these elements should not be limited by these terms. These terms are used to distinguish one element from another. Thus, a first element discussed below could be termed a second element without departing from the teachings of the present inventive concept. As used herein, the term “and/or” includes any and all combinations of one or more of the associated listed items.

The terminology used herein is for the purpose of describing particular exemplary embodiments only and is not intended to be limiting of the present inventive concept. As used herein, the singular forms “a,” “an” and “the” are intended to include the plural forms as well, unless the context clearly indicates otherwise. It will be further understood that the terms “including,” “comprising,” “having,” “containing,” “involving,” and variations thereof, are meant to encompass the items listed thereafter and equivalents thereof, but do not preclude the presence or addition of one or more other features, integers, steps, operations, elements, components, and/or groups thereof. “Consisting of” shall be understood as a close-ended relating to a limited range of elements or features. “Consisting essentially of” limits the scope to the specified elements or steps but does not exclude those that do not materially affect the basic and novel characteristics of the claimed invention.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

“Radiation Treatment Machine” refers to the machine or device that generates various particle flux, externally or locally near tumor, for radiation treatment. Some embodiments include, but not limited to, X ray machines, teletherapy machines incorporating gamma rays, particle accelerators such as cyclotron, microtron and LINAC incorporting photons, electrons or protons, or brachytherapy devices incorporating radionuclide sources.

“Beam” normally refers to the treatment head of treatment machine and the flux of particles that will emit from the treatment head when it is on. The beam can then be characterized by the particle fluence and energy spectrum profile on a reference plane underneath the exit of the treatment head. The spatial distribution of particles emanating from the beam may be further confined by the geometric shape of the one or more collimators.

As used herein, “radiation treatment planning” or “treatment planning” means the process in radiotherapy where a team of radiation oncologists, radiation therapist, medical physicists and medical dosimetrists plan the appropriate external beam radiotherapy or internal brachytherapy treatment technique for a patient with cancer. The resulting plan is called “radiation treatment plan” or “treatment plan”. In treatment planning, various image data are used to form a virtual patient for a computer-aided design procedure. Treatment simulations are used to plan the geometric, radiological, and dosimetric aspects of the therapy using radiation transport simulations and optimization. For intensity modulated radiation therapy (IMRT), this process involves selecting the appropriate beam particle (photons, electron and perhaps protons), energy (e.g. 6 MV, 18 MV) and arrangements, and for each beam selecting a set of machine deliverable MLC segments and their MUs in the case of using static MLC for intensity modulation or selecting the position and velocity of each leaf of the MLC in the case of using dynamic MLC for intensity modulation. For brachytherapy, this process involves selecting the appropriate catheter positions and source dwell times (in HDR brachytherapy) or seeds positions (in LDR brachytherapy). The more formal optimization process is typically referred to as forward planning and inverse planning. Plans are often assessed with the aid of dose-volume histograms, allowing the clinician to evaluate the uniformity of the dose to the diseased tissue (tumor) and sparing of healthy structures. Examples of current Radiation Treatment Planning Systems (RTPS) include ScandiPlan (Scanditronix), ISOgray (DOSIsoft), Monaco (CMS/Elekta), Theraplan Plus (Nucletron), Oncentra-External Beam and Brachy Therapy (Elekta), Pinnacle (Philips Medical systems), Plato RTS & Plato BPS (Nucletron), Corvus (Nomos), Eclipse (Varian), Gammaknife (Elekta), VariSeed-Prostate LDR Brachytherapy (Varian), XKnife (Integra Radionics), RayStation (RaySearch Laboratories) and PlanW (UJP PRAHA a.s.).

“Imaging” or “image data” refers to the technique or associated data generated by, e.g., x-ray computed tomography (CT) which is often the primary image set for treatment planning, magnetic resonance imaging (MRI) which can be the primary or secondary image set for soft tissue contouring, and positron emission tomography (PET) and single photon emission computed tomography (SPECT) which can be used for cases where specific uptake studies can enhance planning target volume delineation.

Specifically, CT scan uses computer-controlled X-rays to create images of the body. An x-ray tube is rotated around the patient. X-rays are emitted by the tube as it transverses around the body. Linear detectors are positioned on the opposite side of the x-ray tube to receive the transmitted x-ray beams after attenuation. Since the x-ray attenuation properties of various tissues differ, the final transmitted x-rays can be correlated to the tissue properties within its path. Detectors will collect the profiles of x-rays with different strength passed through the patient and generate the projection data. Through the backward projection method, the cross-section image slices will be reconstructed from the collected data. CT scan images are three dimensional.

MRI uses radio waves in the presence of a strong magnetic field that surrounds the opening of the MRI machine where the patient lies to get tissues to emit radio waves of their own. Different tissues (including tumors) emit a more or less intense signal based on their chemical makeup, so a picture of the body organs can be displayed on a computer screen. Much like CT scans, MRI can produce three-dimensional images of sections of the body, but MRI is sometimes more sensitive than CT scans for distinguishing soft tissues.

PET scan creates computerized images of chemical changes, such as sugar metabolism, that take place in tissue. Typically, the patient is given an injection of a substance that consists of a combination of a sugar and a small amount of radioactively labeled sugar. The radioactive sugar can help in locating a tumor, because cancer cells take up or absorb sugar more avidly than other tissues in the body such that the radioactive sugar will accumulate in the tumor. A PET scanner is used to detect the distribution of the sugar in the tumor and in the body. In some embodiments, by the combined matching of a CT scan with PET images, there is an improved capacity to discriminate normal from abnormal tissues.

SPECT uses radioactive tracers and a scanner to record data that a computer constructs into two- or three-dimensional images. A small amount of a radioactive drug is injected into a vein and a scanner is used to make detailed images of areas inside the body where the radioactive material is taken up by the cells. SPECT can give information about blood flow to tissues and chemical reactions (metabolism) in the body.

A “point of interest” (POI) is a specific point location inside the phantom or human body that physician, physicist or dosimetrist may find useful or interesting in the procedures of radiation treatment. An example in radiotherapy is the iso-center point which normally locates at the geometric center of the tumor volume and servers as the rotational center of the accelerator gantry.

A “region of interest” (ROI) is a selected subset of samples within a medical dataset identified for a particular clinical purpose. In the context of radiotherapy, it may refer to, in the discretized version, a subset of pixels in a slice of 2d medical image or a subset of voxels in the reconstructed 3d imaging data; or it may refer to, in the continuous version, the area inside the boundary curve in a slice of 2d medical image or the volume inside the boundary surface in the reconstructed 3d imaging data. For example, the ROI in “Gross Tumor Volume” (GTV) is the gross palpable or visible demonstrable extent and location of malignant growth. The GTV is usually based on information obtained from a combination of imaging modalities (computed tomography (CT), magnetic resonance imaging (MRI), ultrasound, etc.), diagnostic modalities (pathology and histological reports, etc.) and clinical examination. The ROI in “Clinical Target Volume” (CTV) is the tissue volume that contains a demonstrable GTV and/or sub-clinical microscopic malignant disease, which has to be eliminated (ICRU Report No. 50). This volume thus has to be treated adequately in order to achieve the aim of therapy, cure or palliation. The CTV often includes the area directly surrounding the GTV, which may contain microscopic disease and other areas considered to be at risk and requiring treatment (e.g. positive lymph nodes). The CTV is an anatomical-clinical volume and is usually determined by the radiation oncologist, often after other relevant specialists such as pathologists or radiologists have been consulted. The CTV is usually stated as a fixed or variable margin around the GTV (e.g., CTV=GTV+1 cm margin), but in some cases it is the same as the GTV (e.g. prostate boost to the gland only). The ROI in “Planning Target Volume” (PTV) is a geometrical concept, and it is defined to select appropriate beam arrangements, taking into consideration the net effect of all possible geometrical variations, in order to ensure that the prescribed dose is actually absorbed in the CTV (ICRU Report No. 50). The PTV includes the internal target margin and an additional margin for set-up uncertainties, machine tolerances and intratreatment variations. The PTV is linked to the reference frame of the treatment machine and is often described as the CTV plus a fixed or variable margin (e.g., PTV=CTV+1 cm). Other ROIs may include the volumes of various organs at risk. The organ at risk is an organ whose sensitivity to radiation is such that the dose received from a treatment plan may be significant compared with its tolerance, possibly requiring a change in the beam arrangement or a change in the dose.

“Forward planning” is a technique used in external-beam radiotherapy to produce a treatment plan. In forward planning, a treatment (e.g., by a dosimetrist) places beams into a radiotherapy treatment planning system which can deliver sufficient radiation to a tumor while both sparing critical organs and minimizing the dose to healthy tissue. The required decisions include how many radiation beams to use, which angles each will be delivered from, whether attenuating wedges be used, and which multileaf collimator configuration will be used to shape the radiation from each beam. Once the treatment planner has made an initial plan, the treatment planning system calculates the required monitor units to deliver a prescribed dose to a specific area in the patient which is dependent on beam modifiers that include wedges, specialized collimation, field sizes, tumor depth, etc. The information from a prior CT scan of the patient allows more accurate modeling of the behavior of the radiation as it travels through the patient's tissues. Different dose prediction models are available, including pencil beam, convolution-superposition and Monte Carlo simulation, with precision versus computation time being the relevant trade-off. This type of planning is used for the majority of external-beam radiotherapy treatments, but is only sufficiently adept to handle relatively simple cases—cases in which the tumor has a simple shape and is not near any critical organs. For more sophisticated plans, inverse planning is used to create an intensity-modulated treatment plan. This is now also used as a part of post-mastectomy radiotherapy (PMRT) planning.

“Inverse planning” is a technique used to design a radiotherapy treatment plan. A radiation oncologist defines a patient's critical organs and tumor then a dosimetrist gives target doses and importance factors for each. Then, an optimization program is run to find the treatment plan which best matches all the input criteria. In contrast to the manual trial-and-error process known in oncology as “forward planning”, “inverse planning” uses the optimizer to solve the Inverse Problem as set up by the dosimetrist. HIPO (Hybrid Inverse Planning & Optimization), developed by Pi-Medical Ltd., is one exemplary algorithm.

“Dose” refers to the amount of radiation used in photon radiation therapy and is measured in gray (Gy), which varies depending on the type and stage of cancer being treated. For curative cases, the typical dose for a solid epithelial tumor ranges from 60 to 80 Gy, while lymphomas are treated with 20 to 40 Gy. Preventive (adjuvant) doses are typically around 45-60 Gy in 1.8-2 Gy fractions (for breast, head, and neck cancers.) Many other factors are considered by radiation oncologists when selecting a dose, including whether the patient is receiving chemotherapy, patient comorbidities, whether radiation therapy is being administered before or after surgery, and the degree of success of surgery. Delivery parameters of a prescribed dose we determined during treatment planning (part of dosimetry). Treatment planning is generally performed on dedicated computers using specialized treatment planning software. Depending on the radiation delivery method, several angles or sources may be used to sum to the total necessary dose. The planner will try to design a plan that delivers a uniform prescription dose to the tumor and minimizes dose to surrounding healthy tissues.

A “dose-volume histogram” (DVH) is a summary of 3D dose distributions in a graphical 2D format, which is a function that describes what fraction of tissue volume has received radiation dose that is greater than some value. For example, in DVH(x)=v, x is dose level and v is the fraction that receives more than x dose. In modern radiation therapy, 3D dose distributions are typically created in a computerized TPS (Treatment Planning System) based on a 3D reconstruction of a CT scan. The “volume” referred to in DVH analysis is a target of radiation treatment, a healthy organ nearby a target, or an arbitrary structure. A DVH used clinically usually includes all structures and targets of interest in the radiotherapy plan, each line plotted a different color, representing a different structure. The vertical axis is almost always plotted as percent volume (rather than absolute volume), as well.

DVHs can be visualized in either of two ways: differential DVHs or cumulative DVHs. A DVH is created by first determining the size of the dose bins of the histogram. Bins can be of arbitrary size, e.g. 0-1 Gy, 1.001-2 Gy, 2.001-3 Gy, etc. In a differential DVH, bar or column height indicates the volume of structure receiving a dose given by the bin. Bin doses are along the horizontal axis, and structure volumes (either percent or absolute volumes) are on the vertical. The differential DVH takes the appearance of a typical histogram. The cumulative DVH is plotted with bin doses along the horizontal axis, as well. However, the column height of the first bin (0-1 Gy, e.g.) represents the volume of structure receiving greater than or equal to that dose. The column height of the second bin (1.001-2 Gy, e.g.) represents the volume of structure receiving greater than or equal to that dose, etc. With very fine (small) bin sizes, the cumulative DVH takes on the appearance of a smooth line graph. The lines always slope and start from top-left to bottom-right. For a structure receiving a very homogenous dose (100% of the volume receiving exactly 10 Gy, for example) the cumulative DVH will appear as a horizontal line at the top of the graph, at 100% volume as plotted vertically, with a vertical drop at 10 Gy on the horizontal axis.

“Intensity-modulated radiation therapy” (IMRT) is an advanced type of high-precision radiation that is the next generation of 3-dimensional conformal radiation therapy (3DCRT), in which the intensity profile of each radiation beam is shaped using a multileaf collimator (MLC) from a beam's eye view (BEV). And a variable number of beams are used together to fit the profile of the target. IMRT also improves the ability to conform the treatment volume to concave tumor shapes, for example when the tumor is wrapped around a vulnerable structure such as the spinal cord or a major organ or blood vessel. Computer-controlled x-ray accelerators distribute precise radiation doses to malignant tumors or specific areas within the tumor. The pattern of radiation delivery is determined using highly tailored computing applications to perform optimization and treatment simulation (Treatment Planning). The radiation dose is consistent with the 3-D shape of the tumor by controlling, or modulating, the radiation beam's intensity. The radiation dose intensity is elevated near the gross tumor volume while radiation among the neighboring normal tissue is decreased or avoided completely. This results in better tumor targeting, lessened side effects, and improved treatment outcomes.

“Volumetric modulated arc therapy” (VMAT) is another X-ray radiation technique that allows the simultaneous variation of three parameters during treatment delivery, i.e., gantry rotation speed, treatment aperture shape via movement of MLC leaves and dose rate. VMAT differs from IMRT because it delivers the dose to the whole volume while the gantry is rotating, rather than from several fixed beams with different angles. Therefore, VMAT is able to provide better plan quality and much faster dose delivery but more complicated optimization and delivery process comparing with IMRT.

“Intensity modulated proton therapy” (IMPT) is a proton based radiation therapy which implies the electromagnetic spatial control of well-circumscribed pencil beams of protons of variable energy and intensity. Proton pencil beams take advantage of the charged-particle Bragg peak—the characteristic peak of dose at the end of range—combined with the modulation of pencil beam variables to create target-local modulations in dose that achieves the dose objectives.

A “remote device” as used herein refers to a device (e.g., desktop, workstation, laptop, pad or mobile device) with a pure web-browser based interface and/or a wireless mobile device with wireless software application interface installed. In some embodiments, the remote device excludes the use of desktop sharing or desktop client/server architecture for traditional remote radiotherapy software.

Embodiments of the present disclosure are generally related to providing optimized radiation therapy treatment plans in an efficient manner. Methods and systems disclosed herein facilitate a user to access and/or obtain an optimized treatment plan from a remote location at any given time, by utilizing a centralized computing platform/server (e.g., a cloud server). In some embodiments, treatment plans can be generated manually by a user using software modules, or be automatically generated on a central server. Alternatively, image, contour and prescription data can also be exported and forwarded to a treatment plan system (TPS), such that treatment plans can be generated at the TPS either manually or automatically and be subsequently forwarded or imported to the central server.

Methods and systems herein may be used to plan various types of radiation treatment modalities suitable for therapy. Some exemplary modalities include IMRT, VMAT, IMPT, or Brachytherapy. For illustrative purposes, IMRT is used in some examples to describe the planning workflow. It should be understood that the same planning methods and systems are equally applicable to other modalities such as VMAT and IMPT.

In some embodiments, a workflow for generating an optimized radiation therapy treatment plan can include the following steps:
(1) A CT (computerized tomography) or MRI (magnetic resonance imaging) machine sends diagnostic images (e.g., images of organs etc.) to a local Digital Imaging and Communications in Medicine (DICOM) server.
(2) The local DICOM server can synchronize the received image data to a mirror node on a cloud server.
(3) A user, such as a physician or physicist, may use a first end user device (e.g., a wireless mobile device) having a processor unit (e.g., a computer or a wireless phone) to retrieve the image data from either a local computing server or a cloud computing server, depending on the user's location.
(4) The user can then use the first end user device to delineate the contours of target/tumor volume and critical organs, one image at a time (e.g., slice by slice), at the user's convenience, using a software program (e.g., a unified web-based Graphical User Interface or GUI) or an app (e.g., a client app compatible with all mobile operating systems (OS's), including the Apple® iOS, Android or the Microsoft Window Phones) in the first end user device. Suitable user interface includes, e.g., mouse and touch screen (by finger or pen). In some embodiments, the contours may also be auto generated by segmentation software on the server or the first end user device, and the user can optionally modify the auto-generated contours manually through an interface (e.g., touch screen).
(5) The user can also use the program or app to input the treatment prescription values (e.g., radiation dose).
(6) Subsequently, contouring data and prescription values may be forwarded to the local server or the cloud server (e.g., the server node on the cloud).
(7) Three-dimensional (3D) volume and surface representation of the target volume and critical organs may be reconstructed from the received two dimensional (2D) slice contours data by the server.
(8) In some embodiments, the server (local or cloud) may be configured to generate and/or optimize a radiation treatment plan.
(9) The plan (e.g., after optimization) may be later retrieved by the user at a remote location, using a second end user device (e.g., a computer or a wireless mobile device). The second end user device can be the same as, or different from, the first end user device.
(10) The user can review and evaluate the plan based on the plan parameters, statistical information or indices that can be auto-calculated by one or more software (e.g., TPS) pre-installed on the server. These include one or more of: beam's eye view (BEV—the view from the perspective of an observer at the radiation source looking out along the radiation axis at the target and normal tissues included in that particular radiation portal), Digitally Reconstructed Radiography (DRR), radiation beam segments, 2D isodose lines, 3D iso surfaces, dose-volume histograms, Conformality Index (CI), Heterogeneity Index (HI) of the target volume, Tumor Control Probability (TCP), and Normal Tissue Complication Probability (NTCP).
(11) After review, the user, using the end user device, can present the treatment plan to a supervisor for approval, along with the user's notes, if any, that were entered by the user via the program or app.
(12) The supervisor may approve the treatment plan, at which time the plan may be sent from the supervisor's end user device or the server to a radiation treatment machine such as a linear accelerator (LINAC) for verification. Once verified, schedule the patient so that the treatment plan may be executed.
(13) In instances where the treatment plan is not approved or is rejected by the supervisor, go to step (4), until a satisfactory plan is generated and approved.

One or more end user devices can be used in the workflow of the present disclosure. The end user devices can be wireless devices. As such, a user equipped with a wireless device can access and modify a treatment plan from almost anywhere, and at any time that's convenient to the user, thereby greatly improving the workflow efficiency. In addition, a centralized cloud server provides for a central data depository for storing a large quantity of radiation treatment plans and relevant treatment data, where such plans and data can be readily accessed from remote locations, thereby providing a ubiquitously accessible data source for radiation therapy clinical research and/or treatment plan data mining. In certain embodiments, access control may be implemented such that only one user at a time is permitted to modify the image data and/or the treatment plan, while optionally permitting “read-only” access by other users.

In some embodiments, the wireless device can be configured to provide notifications to a user regarding status of each step of the workflow process, reminding the user with new tasks that may require user disposition. Notification methods can include means commonly used in the wireless industry, including but not limited to methods such as sounds (e.g., beep or ring), short messages, voice messages, or voice calls, etc.

The remote wireless device can also be configured to provide such functionalities as displaying the iso-dose line of the dose distribution of the treatment plan, the beam eye view plan segments and DRR of each beam, the 2D and 3D dose distribution, the 3D region of interest (ROI) surface and DVH. The device additionally provides “submit”, “reject”, “approve”, “comment” and the like functionalities to enable the progression and/or circulation of the plan in the workflow.

In some embodiments, the server can operate by first collecting the prescription data and the optimization parameters from the user, performing optimization based on the collected data, and returning the treatment plan back to the user A DVH (dose-volume histogram) can also be included to display the statistical information of the dose distribution generated by the treatment plan. If satisfactory to the user, then the treatment plan can be forwarded to the supervisor for approval and/or a radiation treatment machine for execution. If not satisfactory, the user can modify the prescription data and/or the optimization parameters and have the server perform optimization again until an optimal plan is obtained.

In certain embodiments, the server can collect the prescription data, perform fully automatic optimization based the prescription data, and return the treatment plan back to the user. For example, fully automatic planning can be achieved upon one-button click, where the user does not have to enter the objective function parameters at all. The user needs only to enter the prescription data. This is a further improvement on inverse treatment planning.

In various embodiments, data can be transferred between the remote device (e.g., a wireless mobile device) and the server over WiFi or wireless internet using TCP or HTTP. A multiresolution method can be used. First, a lower resolution image fitting the screen of the remote device (e.g., a wireless mobile device such as a smartphone) is sent at the request of the user and when the user requests operation such as zoom, higher resolution image can be requested from the server just in time (JIT). This technique also applies to 3D object data transmission. In some embodiments, the wireless device may be configured to include location-awareness features which will automatically detect its location by trying to connect to a local server (e.g., located within host hospitals) using echo messages. If the device was within the connection range of a local area network (LAN) of a host hospital, the device can be automatically connected to the local server. Alternatively, the device can be connected to a node on the cloud or a central server using TCP, HTTP or HTTPS, or login into a cloud network using VPN first and then access the cloud.

The radiation treatment plan generated by the methods and systems of the present disclosure can include a set of beams. Each beam may comprise radiation beam angle, couch angle and beam energy. In step-and-shoot mode, each beam can further comprise one or more segments. Each segment comprises the left and right leaf position of a group of leafs in the MLC (multi-leaf collimator) and duration of the open time. In dynamic MLC (DMLC) mode, each beam comprises the position and velocity of the left and right leaf of a group of leafs in the DMLC.

For VMAT, the plan can comprise one or more segments, wherein each segment comprises beam rotation direction, start angle, end angle and the beam rotation speed and the position and velocity of the left and right leaf of a group of leafs in the dynamic MLC simultaneously. For IMPT, the plan can comprise a set of beams. Each beam comprises beam angle, couch angle, beam energy and the fluence map of the beam (the intensity distribution of the particle flux or energy in the field of the beam).

Compared to conventional trial-and-error planning which is time consuming and requires well-trained dosimetrists, methods and systems of the present disclosure provide, in some embodiments, autoplan which significantly saves time and cost.

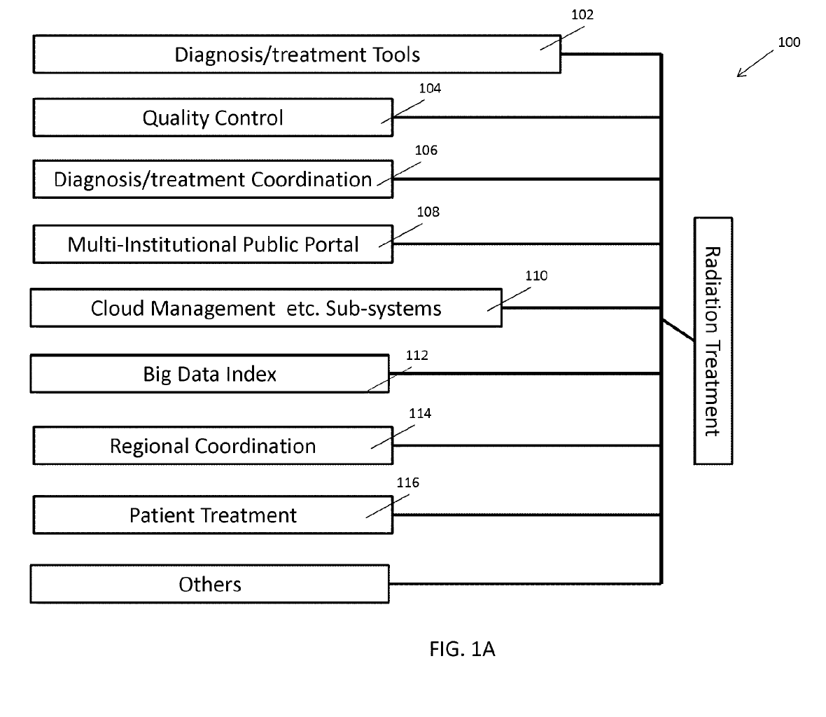

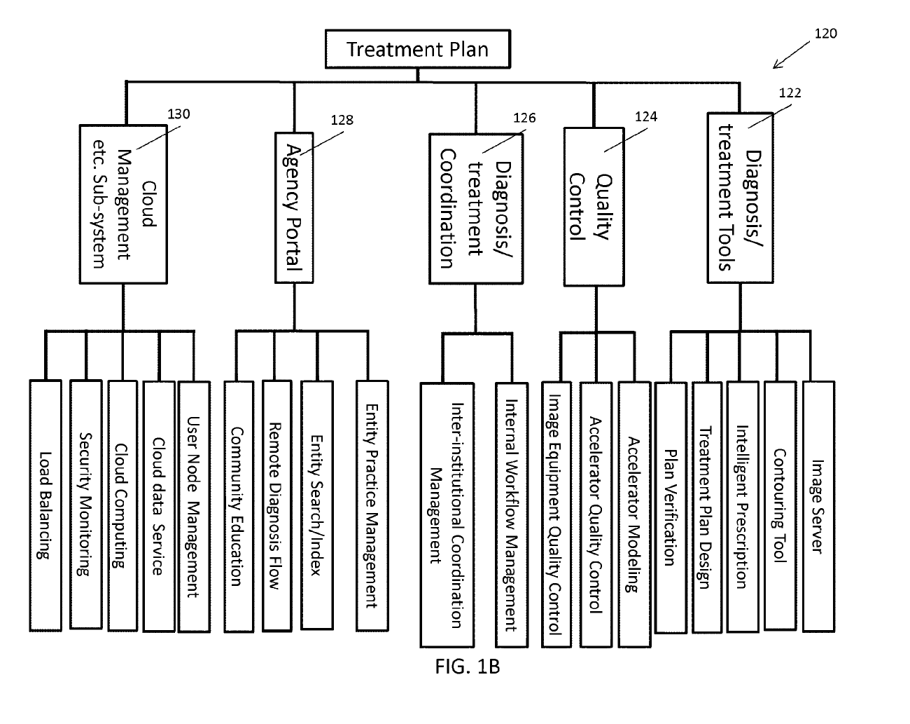

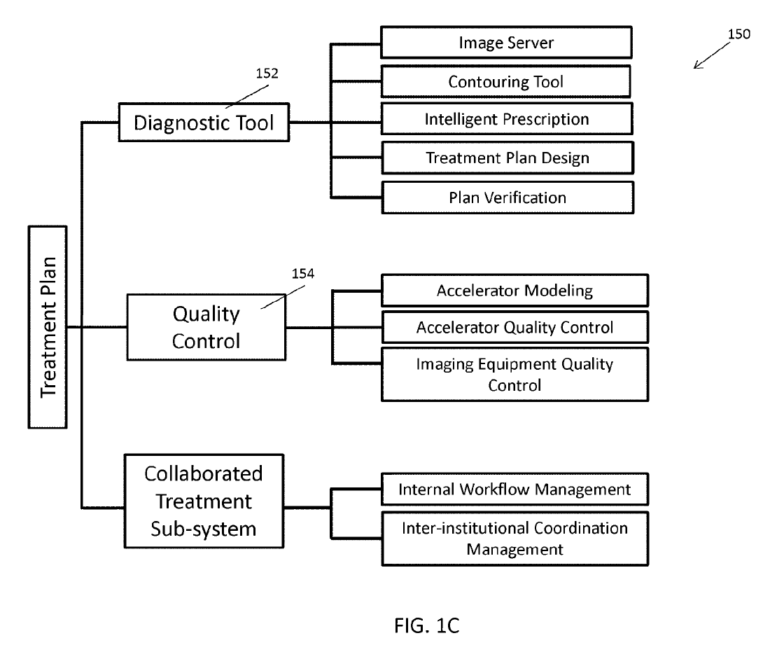

It should be appreciated that a treatment plan generation platform may include various core building blocks/components that accommodate different customer needs. For example, a comprehensive version and an abridged version can be provided. FIG. 1A illustrates an exemplary comprehensive version which is a cloud-based radiation treatment planning platform 100 aimed to function as a high level managerial system for top-level data search and coordination. For example, the cloud-based platform can include a Diagnosis/treatment Tool module 102 designed to manage treatment and diagnostic equipment, which can include image servers, contouring tools, intelligent prescription, treatment plan design module and plan verification module. The cloud based platform 100 can further include a Quality Control module 104 for monitoring the quality of the radiation treatment, which can be configured to model accelerator, monitor accelerator performance and quality control imaging equipment. The cloud based platform 100 can also include (1) a Diagnosis/treatment Coordination module 106 for internal workflow (e.g., within an institution) management and inter-institutional coordination management, (2) an Agency Portal module 108 for entity practice management, entity search, remote diagnosis/treatment workflow, and entity education community, and (3) a Cloud Management module 110 for managing user nodes, cloud data service, cloud computing, data security and system loads etc. Other modules can be added or removed as needed, such as a Big Data Index module 112, a Regional Collaboration module 114, and a Patient Treatment module 116.

FIG. 1B illustrates an abridged version of a cloud-based platform 120 that can be designed to coordinate radiation treatment between multiple hospitals. The platform 120 illustrated in FIG. 1B can include major modules such as a Diagnosis/treatment Tool module 122, a Quality Control module 124 for monitoring the quality of the radiation treatment, a Diagnosis/treatment Coordination module 126, an Agency Portal module 128, and a Cloud Management module 210.

Another cloud-based platform 150 illustrated in FIG. 1C may be configured to serve individual hospitals. The platform 150 in this case can be modified to focus on managing the various equipments involved in the radiation treatment workflow and the qualities of the treatment provided. Accordingly, such platform 150 can include a Diagnosis/treatment Tool module 152 working together with a Quality Control module 154 to ensure the treatment plan is carried out effectively.

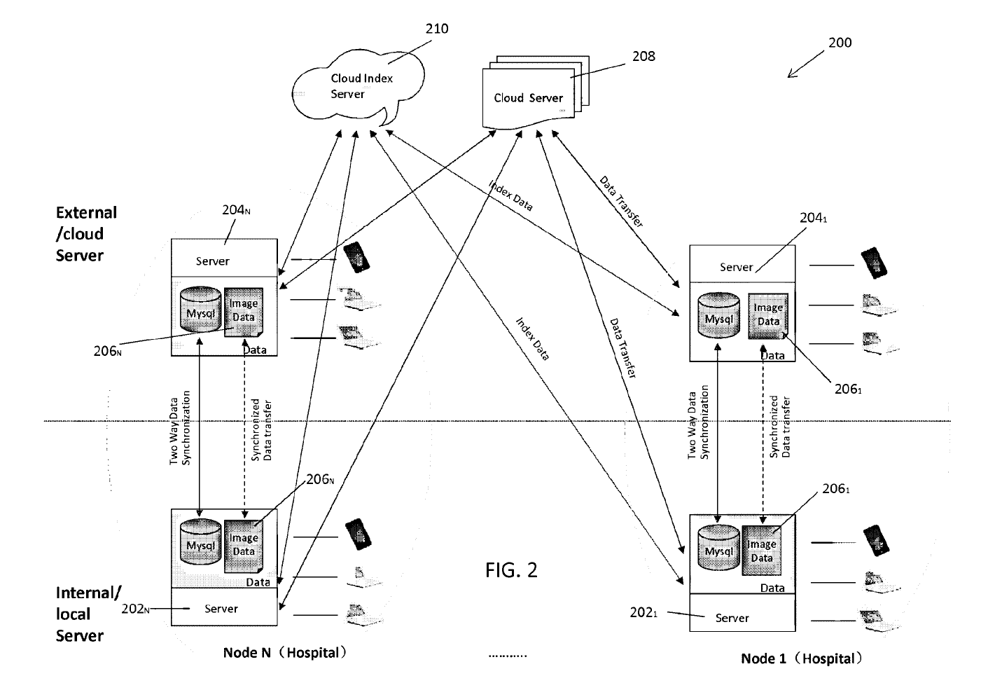

FIG. 2 illustrates a high level overview of a cloud based system 200 for generating a radiation treatment plan in accordance with some embodiments presented herein. As illustrated in FIG. 2, a plurality of local servers, such as servers 202_{1-N} located in hospitals 1 to N can be configured to store CT or MRI generated image data. The image data 206_{1-N} stored on the local servers 202_{1-N} can be accessed by users through end-user devices such as computers or cellular phones. The user can use the end-user devices to review and modify the stored image data 206_{1-N}, such as delineate the contours of target volume and critical organs, one image (i.e., one slice) at a time, at the user's convenience, using a software program or a wireless app readily available in the end user devices. In some embodiments, each of the local servers 202_{1-N} may be further synchronized with a remote server 204_{1-N} located externally to the hospital, where the image data 206_{1-N} can be synchronized and stored onto the external servers 204_{1-N} and are similarly accessible through end-user devices. Image data 206_{1-N} can be copied between the internal 202_{1-N} and external 204_{1-N} servers to ensure the availability and safe keeping of the data 206_{1-N}. In operation, a centralized server node such as a cloud server 208 can collect image data from both the internal 202_{1-N} and/or external 204_{1-N} server for generating optimized treatment plans. In addition, a separate cloud server 210 may be configured to collect and store index data from the internal 202_{1-N} and/or external 204_{1-N} servers, functioning as a centralized index server and providing fast data searches to the users. For example, patient data can be searched through, e.g., a wireless device or a computer, using search parameters such as patient name, age, sex, tumor stage, tumor volume, tumor location and shape, or the vicinity index of tumors to their neighboring organs such as Overlapping Volume Histogram (OVH).

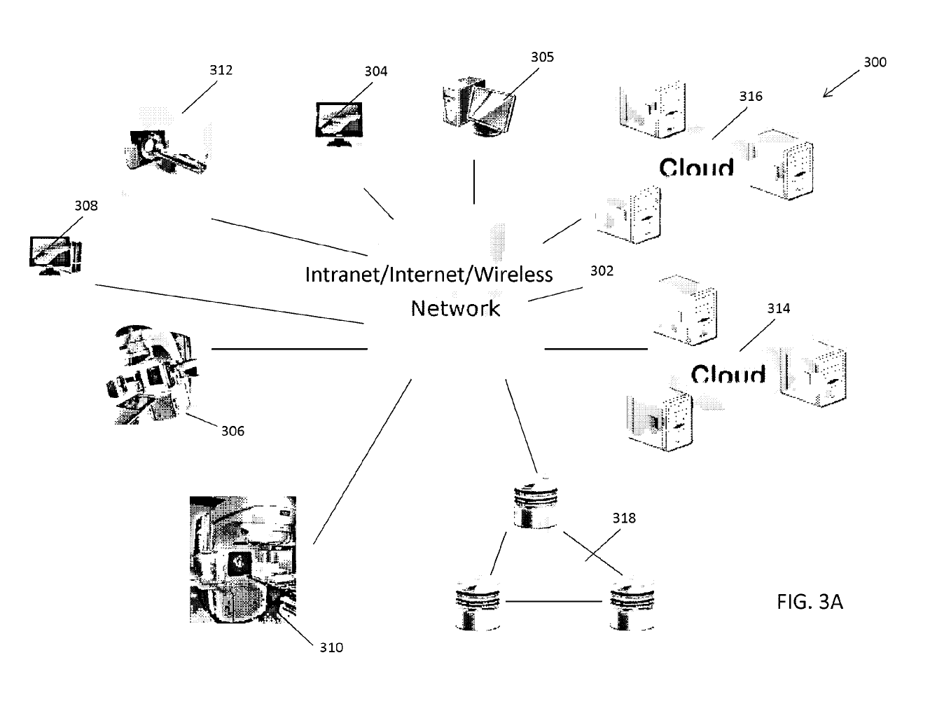

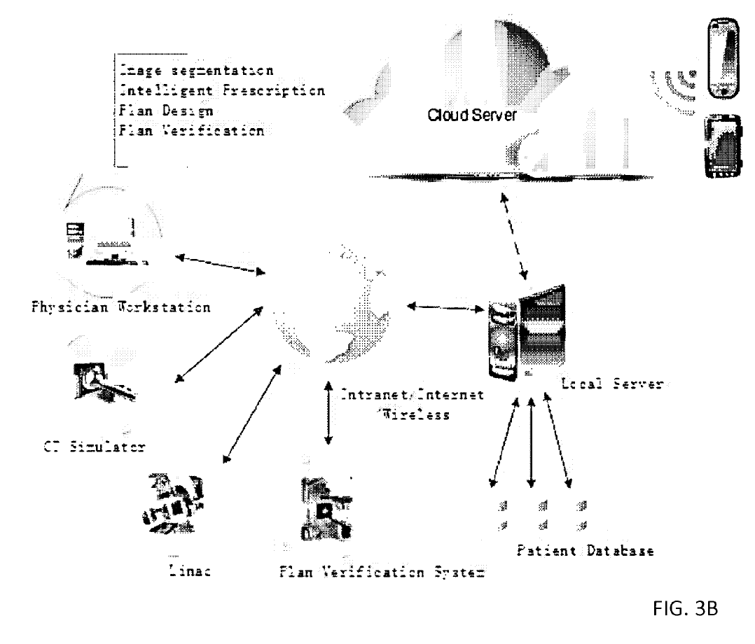

FIG. 3A illustrates another example of a cloud-based system 300 that can be configured to generate and optimize radiation treatment plans. As illustrated in FIG. 3A, a wireless accessible intranet or internet network 302 can be configured to function as a first level depository for storing biometric data such as CT or MRI images. The network 302 may be physically located in proximity to a hospital where the images are collected from, e.g., a CT simulator 312. The network 302 may be connected to and can be accessed by doctor work station 304, workflow server 305, and other end user devices (e.g., computers or wireless mobile devices). In some embodiments, linear accelerator 306 can be directly connected to the network 302 to receive radiation treatment instructions. Furthermore, treatment planning system 308 can be connected to the network 302 to provide treatment plan proposals, which proposals can be verified by treatment plan verification system 310, also connected to the network 302. In some embodiments, the wireless accessible network 302 can function as a midway station for providing and/or receiving biometric data (e.g., CT or MRI images), prescription values, optimization parameters and/or radiation treatment plan data to and from cloud servers. For example, a cloud based decision support system 314 can be connected to the network 302 to provide optimized treatment plans. In some embodiments, the network 302 can be further connected to a cloud-based quality control system 316. In some embodiments, the network 302 can be connected to databases such as patient record database 318, where past radiation treatments and patient history can be readily accessed for reference.

In another embodiment illustrated in FIG. 3B, a wireless device (e.g., smart phone) can be used to access image data, perform contouring, review and/or verify treatment plan, search patient database, etc., while communicating with one or more of the local server, the cloud server, patient database, plan verification system, LINAC, CT simulator, and physician workstation.

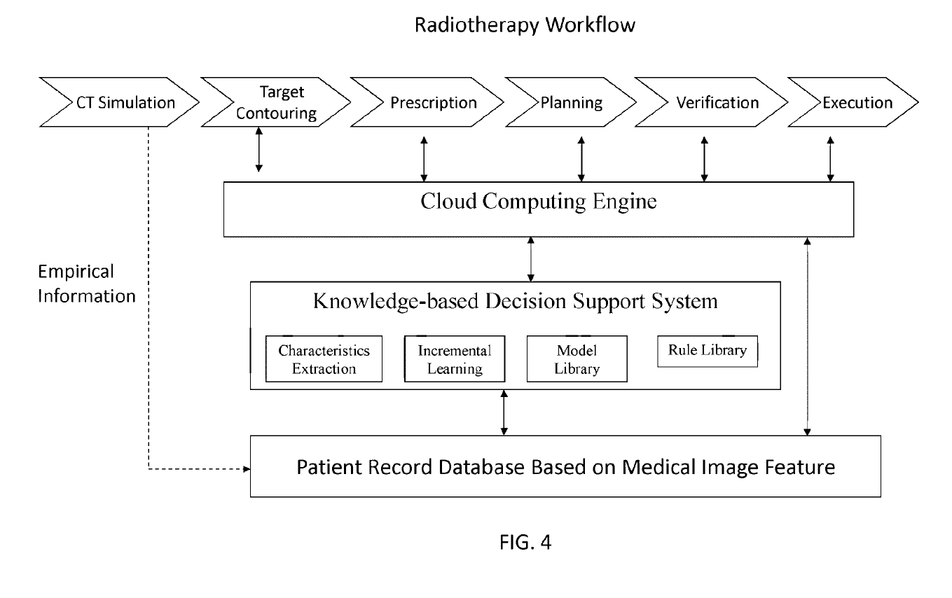

By directly connecting various components to cloud servers, a user not only has access to a vast quantity of radiation treatment related resources such as indexed patient records and pathology consultation, but the cloud-base system can also be configured to perform treatment plan optimization, generate autoplans, display plans anywhere anytime and verify plans with ease. In some embodiments, a cloud based radiation treatment plan generation system may also be designed to allow users to monitor and control the various stages of the treatment workflow using end user devices such as a computer or a wireless phone. As illustrated in FIG. 4, an exemplary radiotherapy workflow of the present disclosure can include six steps, CT simulation, target contouring (e.g., on an end-user device), prescription (e.g., on an end-user device), treatment planning (e.g., on a TPS or a server), plan verification (e.g., on an end-user device) and plan execution. For each of the last four steps, the corresponding device or server can be connected to a cloud computing engine for optimization. The cloud computing engine can be connected to a knowledge-based decision support system that can include various modules, such as image feature extraction, incremental learning, model library and rule library. The decision support system can be connected to a patient record database that can be based on medical image features and be reinforced by CT simulation and empirical information.

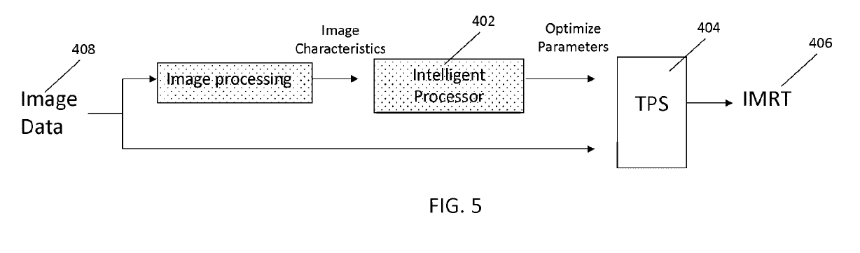

In some embodiments, the methods and systems described herein can be configured to perform radiation treatment plan optimization and then provide delivery modalities (e.g., intensity-modulated radiation therapy (IMRT)) to linear accelerators to provide precise radiation treatment to specific areas. This process is sometimes referred to as “autoplan”. Referring now to FIG. 5, image data 408 supplied by a user (e.g., using CT or MRI machines) can be processed, to extract features from the images and process the features through an intelligent processor 402. The intelligent processor 402 can be configured to optimize treatment parameters such as beam orientations, objective function parameters, or weights. The optimized parameters can subsequently be processed through a treatment planning system (TPS) 404 to generate an IMRT Plan 406 for the accelerators. In some embodiments, the image data 408 can also be supplied to the treatment planning system concurrently with the processed data for generating IMRT plans.

Other treatment modalities such as VMAT or IMPT may also similarly be generated and executed by equipment such as linear or cyclotron accelerators. In some embodiments, a VMAT based treatment plan may include continuous reshaping and changing the intensity of the radiation beam as a linear accelerator moves around the body. In some other embodiments, with an IMPT based radiation treatment plan, precision, depth and intensity of a proton beam may be adjusted by an oncologist or controlled by a computer to trace the peaks and valleys of complex spiderlike tumors while avoiding healthy tissues.

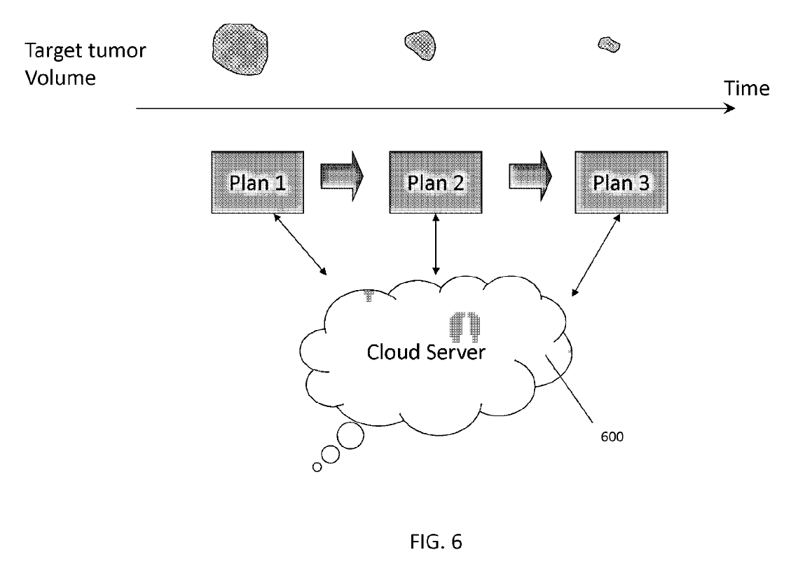

In addition to treatment plan optimization before the start of the first treatment, treatment plans can be adjusted over time to adapt to the changes in, for example target tumor volume. FIG. 6 illustrates a cloud computing server 600 generating treatment plans as tumor sizes changes over time. When tumor volume decreases over time as shown in FIG. 6, new or additional plans (e.g., plan 2, plan 3, etc.) can be generated or optimized by the cloud server 600 to achieve the best possible treatment result.

To minimize radiation dosage to adjacent normal cells, targeted tumors or volumes must be precisely identified. Tumor contouring attempts to achieve that goal with high accuracy and reliability by utilizing various automated segmentation processes. In most cases, contouring is carried out manually by a specialist Digital images, obtained from modalities such as CT or MRI, are used to view and locate the tumor. The physician then marks the boundary of the cancerous tissue on each image. However, the accuracy of the boundary markings varies from physician to physician. This subjective variability is further exacerbated by the limits of the medical image. For instance, images containing many kinds of tissues (e.g., dense breast tissue, ducts, and blood vessels) other than tumors, as well as noise, make it difficult to mark the target using just simple edge manual techniques.

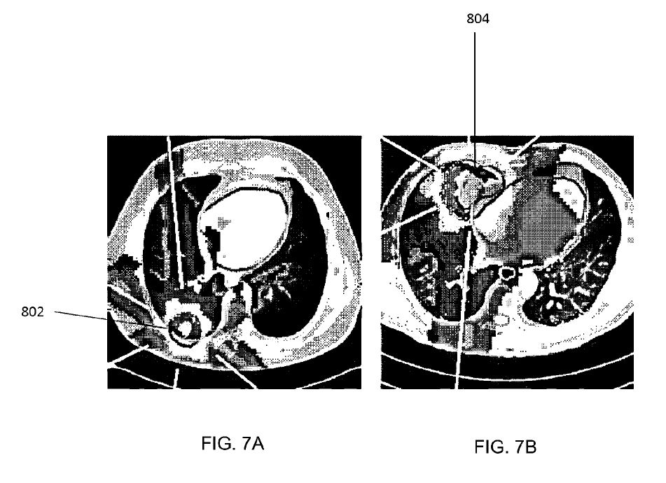

In generating potential radiation treatment plans, radiation target areas or regions of interest may be selected automatically by the server and/or manually by the user. As illustrated in FIGS. 7A and 7B, ROIs 802 and 804 may be selected. Pink outlines indicate heart. Blue area is esophagus and green area is spine.

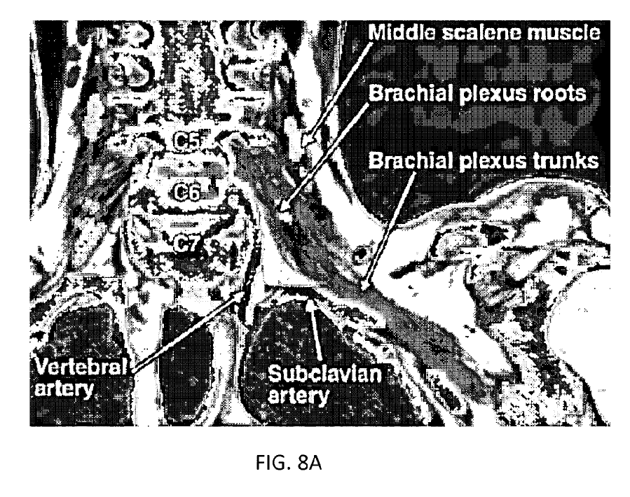

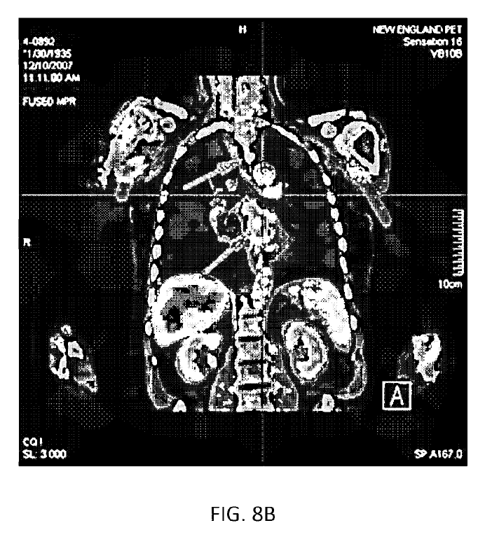

In some embodiments, the server may be configured to automatically outline critical organs from the provided CT or MRI image data. FIG. 8A illustrates an example where a cloud server can be configured to automatically outline anatomic structures such as brachial plexus roots and brachial plexus trunks from a CT or MRI image, which improves the radiation treatment workflow efficiency by eliminate the need for the user to manually identify such structures. The server can have one or more algorithms adapted to recognize tumors and/or critical organs that can self-train via machine learning and/or artificial intelligence.

In some embodiments, as illustrated in FIG. 8B, multiple images may be registered, aligned, superimposed or fused together at the server for diagnostic purposes. For example, CT and PET scan images can be superimposed to improve capacity and accuracy to discriminate normal from abnormal tissues. In another example, as radiation treatment progresses, tumors may change in size and the patient may experience weight loss. In such cases, images taken at different stages of treatment may be aligned together to give physicians an overview of the anatomic changes that have occurred to date so that they can adjust radiation treatment plan accordingly. This is also called “image registration” which is the process to find the best alignment to map or transform the points in one image set to the points of another image set. Registration can be rigid or nonrigid. Rigid body and affine transformation define rigid transformation in which the transformed coordinates are the linear transformations of the original coordinates. Registration for data of the same patient taken at different points in time such as change detection or tumor monitoring often involves nonrigid or elastic registration to cope with deformation of the subject (due to breathing, anatomical changes, and so forth). Nonrigid registration of medical images can also be used to register a patient's data to an anatomical atlas.

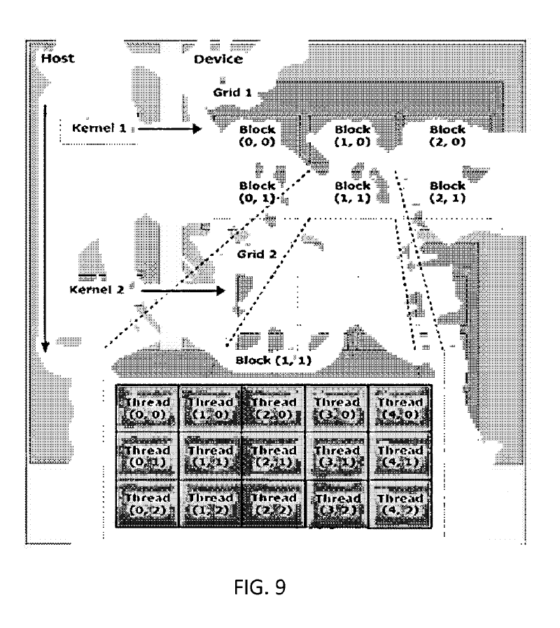

In some embodiments, as illustrated in FIG. 9, specialized Graphics Processing Unit (GPU) may be utilized at the server to optimize treatment plan generation. For example, a specialized GPU may be adopted to perform dose computations, to significantly improve server efficiency.

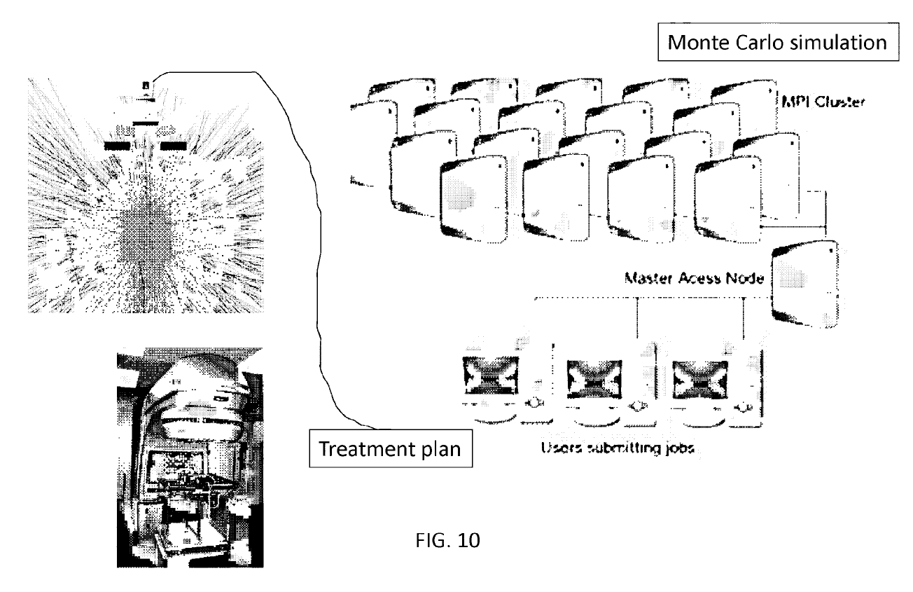

Once a treatment plan has been generated, the server may utilize various means to verify and/or optimize the treatment plan. For example, as illustrated in FIG. 10, Monte-Carlo algorithms may be adopted by the server to verify the treatment plan. Monte Carlo modeling is a statistical method that calculates the dose deposited in the region as a whole by simulating the passage of each photon through the region of interest. In some embodiments, actual beam delivery, including static multileaf collimator (sMLC) or dynamic multileaf collimator (DMLC) may be simulated at the server, thereby eliminating the need for laborious on-site verification using LINAC and phantom.

In treatment planning system (TPS), since the optimization of the machine parameters such as beam directions, MLC aperture and monitor unit, requires many iterations of dose calculation, approximation algorithms are usually involved to speed up the computation. In order to verify the correctness of the final dose distribution and that it falls within a reasonable range of computational error, dose verification procedure can be performed. This can be done using, for example, third party software, where the user can adopt, e.g., a Monte Carlo based dose calculation engine to re-compute the dose distribution based on the machine parameters in the plan, and confirm whether the result agrees well with the dose distribution calculated from the TPS. Alternatively, dosimetric measurement can be used where the user can use the plan to irradiate a water phantom on the bed using the accelerator 2D detectors are installed in the phantom. After irradiation, 2D dose distribution data can be measured and read out using specialized software. The 2D dose distribution data can be matched with the dose distribution in water calculated from the same plan using TPS.

In some embodiments, radiation prescription data and optimization parameters can be firstly entered by a user using web GUIs or wireless apps to a local area network, where such data and parameters can be subsequently synchronized to a cloud server for processing (e.g., optimization). The web GUI or wireless app allows the user to access and modify patient information at a cloud server for generating and optimizing radiation therapy treatment plans tailored. For example, imaging equipment such as a CT or MRI machine can firstly send diagnostic images to a local server (e.g., a DICOM server), where the local server can synchronize with and upload the images to a cloud server. Once uploaded, the user can access or modify the image data at the local server or the cloud server. The user may, using the web GUI or wireless app, collect information from the images, or modify the image data such as delineate the contour of target volume and critical organs, one slice at a time. In some embodiments, the web GUI or wireless app also allows the user to input treatment prescription values and optimization parameters such as target volume dose and a set of constraints for critical organs to protect (e.g., mean dose value, max cord dose value, etc.). The cloud server can reconstruct a 3D volume and surface representation of the target volume and critical organs from the 2D slice contours data. The server can subsequently generate and optimize a treatment plan based on the contour data and the prescription values. The optimized plan can be accessed by the user from a remote location using the web GUI or wireless app. The optimized plan can also be forwarded to a third party (e.g., supervising physician) for review through the web GUI or wireless app.

In some embodiments, webpages on a computing device or an wireless app can be used to connect to and access the server, depending on the location of the user and/or availability of the device. The webpage or app can provide a list displaying patient names and corresponding information such as illness types, and individual patients may be selected through the list. Once a patient is selected from the list, patient information maybe displayed on a screen, where the screen can have a plurality of tabs for accessing or modifying image data. For example, there can be a tab for displaying point of interests (POI) of an image, a tab for displaying and adjusting region of interest (ROI, such as the volume of the tumor) of the image, a tab for displaying and adjusting radiation beams, a tab for evaluating and optimizing the generated treatment plan, and also a tab for displaying the DVH. It should be appreciated that the exact arrangement and contents of the tabs can be altered so long as radiation therapy data desirable to the user can be displayed and accessed through the webpages or app.

After optimization parameters (e.g., including radiation beam orientations and intensities) have been selected by the user, they may be uploaded to the server. Through the webpage or app, the user has the option to update a current treatment plan or request a new treatment plan. At the server, the treatment plan may be optimized for a single objective or multiple objectives. For the single objective optimization, weight parameters may be assigned to each original objective (e.g., dose distribution, region of interest, etc.), and all the weighted objectives can be summed up to form a single cost function for optimization. For multi-objective optimization, multiple cost functions will be considered. After an optimization process has been completed at the server, a dose-volume histogram (DVH) may be produced to provide statistical information to the user. In a typical DVH diagram, the Y component of each data point on the DVH curve can be defined as the percentage volume of the ROI that receives dose higher than the X component of the data point. This DVH curve may be accessed by other parties (e.g., supervisory physician) to review the statistical information, such as dose distributions inside each ROI, on the assumption that the generated plan is executed accordingly on the accelerators. If the indices reflected by the DVH are satisfactory to the user and relevant third parties, its underlying dose distribution is assumed to be acceptable and so is the treatment plan. Otherwise, the parameters may be modified and the plan re-optimized. In addition, statistical information regarding similar treatment plans may be searched and displayed through the webpage or app.

The webpages or an app associated with a wireless device (e.g., smartphone) may provide a user ubiquitous access to radiation treatment plans at any time. It should be appreciated that such wireless app can be made available to all wireless operating systems (e.g., Android, iOS, Windows Phone, etc.), and as such, treatment data can be accessed and shared among all types of wireless devices.

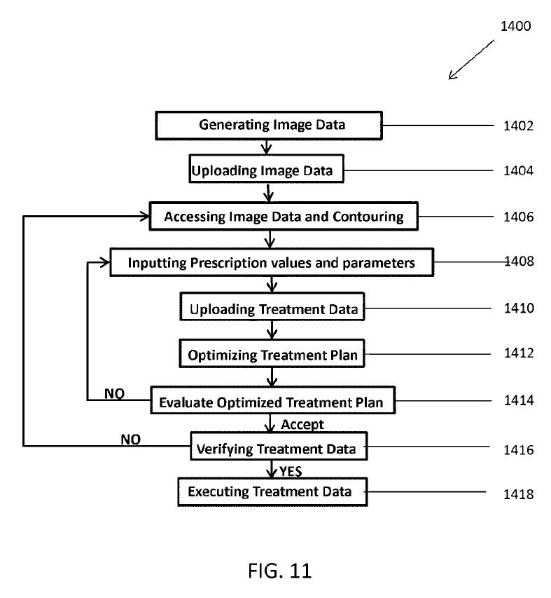

FIG. 11 is an illustration of an exemplary process 1400 in accordance with some embodiments presented herein for generating an optimized radiation treatment plan. In some embodiments, the process 1400 may be used for a computer product having computer program code stored on a non-transitory computer readable medium for generating radiation treatment plans. The process 1400 can start at step 1402. At step 1402, image data for radiation may be generated by imaging equipment such as CT or MRI machines. Subsequently, at step 1404, the image data may be uploaded to a local server and/or a cloud server. Once uploaded, the image data may be accessed by a user through a wireless device or a webpage to review and perform contouring, as stated in step 1406. The user may provide prescription values and/or optimization parameters based on the image data, at step 1408. Then at step 1410 the treatment data may be uploaded to the local and cloud servers. The cloud server may perform optimization to generate an optimized treatment plan, as stated in step 1412. At step 1414, the user can access and evaluate the optimized treatment plan and associated treatment data using the wireless app or webpage from a remote location at any time, and can optionally send it back to, e.g., step 1408 for further optimization if not satisfactory. Subsequently, at step 1416, the optimized treatment data may be forwarded to a third party (e.g., supervising physician) for approval. The third party can likewise review and modify the treatment plan from a remote location at any time using the webpage or the wireless app from their own devices (e.g., computer or smart phone). If the treatment plan is approved, the treatment plan can be executed on an accelerator, as stated in step 1418. Otherwise, the process may be repeated at step 1408 to generate another treatment plan.

It should be appreciated that the process 1400 is exemplary only, and it is understood that other embodiments may add, rearrange, omit, or modify one or more actions.

While the present disclosure has been described with reference to certain embodiments thereof, it should be understood by those skilled in the art that various changes may be made and equivalents may be substituted without departing from the true spirit and scope of the disclosure. In addition, many modifications may be made to adapt to a particular situation, indication, material and composition of matter, process step or steps, without departing from the spirit and scope of the present disclosure. All such modifications are intended to be within the scope of the claims appended hereto.

### INCORPORATION BY REFERENCE

All publications, patents and sequence database entries mentioned herein are hereby incorporated by reference in their entireties as if each individual publication or patent was specifically and individually indicated to be incorporated by reference.

What is claimed is:

1. A method for radiation treatment planning, comprising:
collecting at a central server image data of a tumor and surrounding anatomic structures;
accessing the image data from a remote device, the remote device having an interface for processing the image data into contours of tumor target volume and critical organs;
providing, via the interface of the remote device, a radiation prescription value based on the image data;
processing, at the central server, the contours and the prescription value to generate a radiation treatment plan; and
receiving the radiation treatment plan at the remote device.

2. The method of claim 1, wherein the remote device is a device with a pure web-browser based interface and/or a wireless mobile device.

3. The method of claim 1 or 2, wherein the collecting step comprises collecting the image data from one or more of computerized tomography (CT), positron emission tomography (PET), ultrasound, single-photon emission computed tomography (SPECT) or magnetic resonance imaging (MRI) machine, wherein preferably the collecting step further comprises uploading the image data to a local server that synchronizes with a mirror node on the central server, and wherein more preferably the central server is a cloud server for storing index information associated with the radiation treatment plan, wherein the cloud server is connected to the remote device, allowing access to the cloud server through the interface.

4. The method of claim 1 or 2, wherein the prescription value comprises one or more of radiation dose, hard constraint of the critical organs' dose-volume histogram (DVH), maximal dose limit, minimal dose limit, mean dose limit, and effective uniform dose (EUD).

5. The method of claim 1 or 2, wherein the accessing step comprises generating contours of tumor target volume and critical organs in the image data via the interface.

6. The method of claim 1 or 2, wherein the interface is configured to add to, edit or remove from, the image data a region of interest (ROI).

7. The method of claim 1 or 2, wherein the interface is configured to add to, edit or remove from, the image data a point of interest (POI).

8. The method of claim 1 or 2, wherein the interface is configured to add to, edit or remove from, beam information, wherein the beam information comprises beam angle, couch angle, collimator angle and collimator field size in two dimensions.

9. The method of claim 1 or 2, wherein the accessing step further comprises providing a contouring input device selected from one or more of a finger, a pen and a mouse.

10. The method of claim 1 or 2, wherein in the accessing step, operations supported comprise one or more of zoom in, zoom out, select, move, copy, paste, cut object, resize point of interest (POI), region of interest (ROI) contour object, and change medical image contrast.

11. The method of claim 5, wherein the generating step comprises auto-generating the contours using an automatic segmentation software and modifying the auto-generated contours via the interface.

12. The method of claim 1 or 2, wherein the processing step comprises reconstructing 3D volume and surface representation of the target volume and critical organs.

13. The method of claim 1 or 2, further comprising generating, based on the treatment plan, an evaluation index selected from one or more of, or any combination thereof: two dimensional or three dimensional isodose distribution and/or curve in a region of interest, a dose-volume histogram (DVH) for the tumor target volume and critical organs contoured, Conformality Index (CI), Heterogeneity Index (HI) of the target volume, Tumor Control Probability (TCP), and Normal Tissue Complication Probability (NTCP).

14. The method of claim 12, further comprising forwarding the treatment plan to a third party remote device for approval, together with the evaluation index.

15. The method of claim 13, further comprising notifying the third party remote device by one or more of: highlighted message, instant messaging tool, beep, short recorded sound track, automatic phone call and voice mail.

16. The method of claim 13, further comprising transmitting the approved treatment plan to a radiation treatment machine for execution to carry out a radiation modality.

17. The method of claim 16, wherein the radiation modality is selected from intensity-modulated radiation therapy (IMRT), volumetric modulated are therapy (VMAT), intensity modulated proton therapy (IMPT) and brachytherapy.

18. The method of claim 1 or 2, wherein the processing step comprises generating the radiation treatment plan using a software module.

19. The method of claim 1 or 2, wherein the processing step comprises exporting the contours to a third party treatment planning system (TPS) to generate the radiation treatment plan.

20. A system having computer program code stored on a non-transitory computer readable medium for generating a radiation treatment plan comprising:
a central server having a processor unit for storing and processing image data of a tumor; and
a remote device connected to the central server, the remote device having an interface for accessing the image data and processing the image data into contours of tumor target volume and critical organs, wherein the interface is configured to receive a treatment prescription value and transmit the prescription value to the central server;
wherein the remote device is configured to interact with the central server from a remote location, and wherein the central server has one or more algorithms for processing the contours and the prescription value to generate a radiation treatment plan.

21. The system of claim 20 wherein the remote device is a device with a pure web-browser based interface and/or a wireless mobile device.

22. The system of claim 20 or 21 wherein the central server is a cloud server for storing index information associated with the radiation treatment plan, wherein the cloud server is connected to the remote device, allowing access to the cloud server through the interface.

23. The system of claim 20 or 21, further comprising an imaging equipment for generating the image data, wherein preferably the imaging equipment comprises one or more of a computerized tomography (CT), a positron emission tomography (PET), an ultrasound, a single-photon emission computed tomography (SPECT) and a magnetic resonance imaging (MRI) machine.

24. The system of claim 20 or 21, further comprising a local server for storing the image data, wherein the local server is connected to the remote device and accessible through the interface, and wherein preferably content of the local server is synchronized with the central server.

25. The system of claim 20 or 21, wherein the interface is configured to add to, edit or remove from, the image data a region of interest (ROI).

26. The system of claim 20 or 21, wherein the interface is configured to add to, edit or remove from, the image data a point of interest (POI).

27. The system of claim 20 or 21, wherein the interface is configured to add to, edit or remove from, beam information, wherein the beam information comprises beam angle, couch angle, collimator angle and collimator field size in two dimensions.

28. The system of claim 20 or 21, wherein the prescription value comprises one or more of radiation dose, hard constraint of the critical organs' dose-volume histogram (DVH), maximal dose limit, minimal dose limit, mean dose limit, and effective uniform dose (EUD).

29. The system of claim 20 or 21, wherein the central server is configured to reconstruct three dimensional volume and surface representation of the target volume and critical organs.

30. The system of claim 20 or 21, further comprising a contouring input device selected from one or more of a finger, a pen and a mouse.

31. The system of claim 20 or 21, wherein the remote device supports one or more operations selected from zoom in, zoom out, select, move, copy, paste, cut object, resize point of interest (POI), region of interest (ROI) contour object, and change medical image contrast.

32. The system of claim 20 or 21, wherein the central server is configured to generate, based on the treatment plan, an evaluation index selected from one or more of, or any combination thereof: two dimensional or three dimensional isodose distribution and/or curve in a region of interest, a dose-volume histogram (DVH) for the tumor target volume and critical organs contoured, Conformality Index (CI), Heterogeneity Index (HI) of the target volume, Tumor Control Probability (TCP), and Normal Tissue Complication Probability (NTCP).

33. The system of claim 32, wherein the central server is further configured to forward the treatment plan to a third party remote device for approval, together with the evaluation index.

34. The system of claim 33 further comprising a radiation treatment machine for receiving and executing the approved treatment plan to carry out a radiation modality.

35. The system of claim 34, wherein the radiation modality is selected from intensity-modulated radiation therapy (IMRT), volumetric modulated are therapy (VMAT), intensity modulated proton therapy (IMPT) and brachytherapy.

36. The system of claim 20, 21, 33, 34 or 35, further comprising a third party remote device for reviewing and approving the radiation treatment plan.

37. The system of claim 36, wherein the third party remote device comprises a notification function selected from one or more of: highlighted message, instant messaging tool, beep, short recorded sound track, automatic phone call and voice mail.

38. The system of claim 20 or 21, wherein the central server comprises a software module for generating the radiation treatment plan.

39. The system of claim 20 or 21, further comprising a third party treatment planning system (TPS) for generating the radiation treatment plan.
